(19) United States
(12) Patent Application Publication
(10) Pub. No.: US 2004/0096414 A1
(43) Pub. Date: May 20, 2004

(54) Skin care composition

(75) Inventors: Kiyoaki Mori, Shiga (JP); Hidekazu Tanaka, Hyogo (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/705,327

(22) Filed: Nov. 10, 2003

Related U.S. Application Data
(63) Continuation of application No. PCT/US01/15045, filed on May 10, 2001.

(51) Int. Cl. A61K 7/06; A61K 7/11

(52) U.S. Cl. 424/70.16

(57) ABSTRACT

Disclosed is a skin care composition comprising by weight: (1) from about 0.01% to about 5% of a carboxylic acid/carboxylate copolymer; (2) from about 0.1% to about 2% of a surfactant system comprising two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides; (3) from about 0.05% to about 5% of a silicone component; (4) from about 0.01% to about 5% of an emollient oil; (5) from about 0.1% to about 10% of a sebum absorbing agent; (6) from about 1% to about 20% of a water soluble humectant; and (7) an aqueous carrier; wherein the weight ratio of the surfactant system to the emollient oil is from about 2:1 to about 1:1; and wherein a base composition consisting essentially of components (1) through (4), (6) and (7) above has an absorbance of no more than about 2 at a wave length of 340 nm.

### TECHNICAL FIELD

The present invention relates to a transparent or translucent skin care composition having improved shear thinning property and sebum control performance. The present invention also relates to a method of preparing a stable transparent skin care composition.

### BACKGROUND

Many personal care products currently available to consumers are directed primarily to improving the health and/or physical appearance of the skin. Among these skin care products, many are directed to delaying, minimizing or even eliminating skin wrinkling and other histological changes typically associated with skin aging or environmental damage to human skin.

In order to maintain or return skin to a healthy and/or youthful state, the skin is typically treated with a moisturizing agent. Known moisturizing agents include, for example, water soluble humectants such as glycerin. Increasing the level of moisturizing agent applied to the skin typically provides improved moisturization of the skin. Addition of oily components are also known to be advantageous, in that they provide softness, smoothness, and supplement of intercellular lipid.

Transparent forms of lotions and emulsions incorporating water soluble humectants and oils, to provide moisturization and softness to the skin, are known in the art such as PCT publications WO99/49841, WO00/37029, WO00/61098, and WO00/61083. The transparent appearance is appealing to the consumer, as there is a belief that application of such composition will make their skin transparent and young-looking. It would be even more appealing to the consumer if such product actually provided a feel to the skin as though water were being applied. Water is known to have very low viscosity. When a lotion or emulsion is applied to the skin, the consumer typically rubs the product in the skin or palm of hands. Thus, a composition having shear thinning property is desired in order to provide such water like feel to the skin when applied.

In another aspect, further improvement is desired for providing sebum control to such composition. In yet another aspect, further improvement is desired to improve reduction of tacky and greasy feeling to the skin, particularly when skin treatment agents which imply tacky feeling, such as panthenol and niacinamide, are incorporated in the composition.

Based on the foregoing, there is a need for a transparent or translucent skin care composition having improved shear thinning property and sebum control performance. There is further a need for such a composition having improved reduction of tacky and greasy feel to the skin, even when skin treatment agents are included. There is further a need for a method of preparing a stable transparent skin care composition.

None of the existing art provides all of the advantages and benefits of the present invention.

### SUMMARY

The present invention is directed to a skin care composition comprising by weight:

(1) from about 0.01% to about 5% of a carboxylic acid/carboxylate copolymer;

(2) from about 0.1% to about 2% of a surfactant system comprising two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides;

(3) from about 0.05% to about 5% of a silicone component;

(4) from about 0.01% to about 5% of an emollient oil;

(5) from about 0.1% to about 10% of a sebum absorbing agent;

(6) from about 1% to about 20% of a water soluble humectant; and

(7) an aqueous carrier;

wherein the weight ratio of the surfactant system to the emollient oil is from about 2:1 to about 1:1; and wherein a base composition consisting essentially of components (1) through (4), (6) and (7) above has an absorbance of no more than about 2 at a wave length of 340 nm.

The present invention is also directed to a method of preparation of a composition comprising the steps of:

(a) preparing a first mixture comprising the steps of:

(a1) mixing an emollient oil with a surfactant system and water at a temperature of about 70-80° C.; the amount of such water being from about 0.5 to about 5 times the total weight of the emollient oil and surfactant system;

(a2) cooling the product of (a1) to about 50° C.;

(a3) adding the product of (a2) to water, the temperature of such water being cold enough to immediately cool the obtained mixture to a temperature of no higher than 40° C.;

(b) preparing a second mixture comprising the steps of:

(b1) dispersing a carboxylic acid/carboxylate copolymer into water at a temperature of about 70-80° C. and mixing until homogeneous;

(b2) adding a silicone component to the product of step (b1); and

(c) mixing the first mixture and the second mixture;

wherein the surfactant system comprises two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”.

All percentages, parts and ratios are based upon the total weight of the compositions, of the present invention, unless otherwise specified. All such weights as they pertain to listed ingredients are based on the active level and, therefore, do not include carriers or by-products that may be included in commercially available materials.

Composition

The composition of the present invention is suitable for topical use on human body skin, particularly suitable for facial skin. The composition has a transparent to translucent appearance, and thus is particularly suitable for a clear lotion product.

The present composition provides good moisturizing effect, good spreadability, improved shear thinning property, and sebum control performance. Application to the skin of at least about 4 weeks of the present composition provides significant sebum control. Typically, applications would be on the order of about once per day over such extended periods, while application rates can be up to about three times per day or more. The present composition is stable in regular storage conditions.

The present composition contains practically water-insoluble components, particularly oils, in the form of a microemulsion. The “base composition” of the present composition has high transparency. By “base composition” what is meant is the composition of the present invention containing only the essential components but lacking the sebum absorbing agent, i.e.; a composition consisting essentially of the carboxylic acid/carboxylate copolymer, the surfactant system, the silicone component, the emollient oil, the water soluble humectant, and the aqueous carrier. The base composition has an absorbance of less than about 2 at a wave length of 340 nm; preferably from about 1 to about 1.5 at the same wave length. Absorbance herein can be determined by using the Micro Plate Reader by TECAN Co. Ltd. Herein, “microemulsion” means an emulsion containing water as the continuous phase, in which water-insoluble components, which would otherwise provide a non-transparent appearance, are dispersed as such small droplets that they appear as a transparent emulsion. Preferably, the droplets present in a microemulsion have a mean particle size of less than about 100 nm; more preferably less than about 80 nm. The size of the droplets can be determined using a Laser Scattering Particle Size Distribution Analyzer LA-910 by Horiba (Japan). The size of droplets is measured with the emulsion at a temperature of 25° C.+/−1° C., after 30 seconds mixing. One skilled in the art understands certain silicone components may appear transparent even when dispersed at a larger droplet size. In the present invention, the droplet size of emollient oils and silicone components that do not appear transparent at larger droplet size, are critical for providing the base composition transparent. Without being bound by theory, it is believed the selection of species and amount of the surfactant system provides a suitable microemulsion for dispersing the emollient oils, while also dispersing the silicone components. The weight ratio of the surfactant system to the emollient oil is from about 2:1 to about 1:1.

Depending on the type and amount of sebum absorbing agent included in the present composition, the composition may become less transparent and slightly opaque. Preferably, the type and amount of sebum absorbing agent is selected so that the composition is kept translucent, i.e., not milky or turbid.

Carboxylic Acid/Carboxylate Copolymer

The compositions of the present invention comprise from about 0.01% to about 5%, preferably from about 0.04% to about 1% of a carboxylic acid/carboxylate copolymer. Without being bound by theory, the carboxylic acid/carboxylate copolymer is believed to provide a shear thinning property to the present composition. What is meant by shear thinning property is that a yield point exists within a typical shear stress applicable by the hand on the skin, and that the viscosity of the composition beyond the yield point significantly decreases to the extent such decrease is noticeable by the consumer. In a preferred embodiment, the composition of the present composition, prior to application of shear, has a suitable viscosity, preferably from about 100 mPa∘s to about 6000 mPa∘s, more preferably from about 500 mPa∘s to about 4500 mPa∘s, still preferably from about 700 mPa∘s to about 4000 mPa∘s. The viscosity herein can be suitably measured by Brookfield LV at 20 rpm at 25° C. using either spindle #4, 5, 6 or 7 depending on the viscosity and the characteristic of the composition. In a highly preferred embodiment, the viscosity beyond the yield point is similar to that of water. The shear thinning property provides a low viscous composition directly upon use, thereby providing the feel of applying water to the skin.

Additionally, the carboxylic acid/carboxylate copolymer is capable of dispersing and stabilizing the emollient oils and silicone components in an aqueous environment, so that such components do not separate out. The carboxylic acid/carboxylate copolymer also keeps the composition relatively transparent and at a suitable viscosity without making the composition tacky or greasy upon use.

The carboxylic acid/carboxylate copolymers herein are hydrophobically-modified crosslinked coplymers of carboxylic acid and alkyl carboxylate, and have an amphiphilic property. These carboxylic acid/carboxylate copolymers are obtained by copolymerizing 1) a carboxylic acid monomer such as acrylic acid, methacrylic acid, maleic acid, maleic anhydride, itaconic acid, fumaric acid, crotonic acid, or α-chloroacrylic acid, 2) a carboxylic ester having an alkyl chain of from 1 to about 30 carbons, and preferably 3) a crosslinking agent of the following formula:

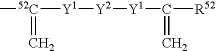

wherein R⁵² is a hydrogen or an alkyl group having from about 1 to about 30 carbons; Y¹, indepedently, is oxygen, CH₂O, COO, OCO,

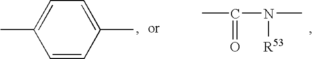

wherein R⁵³ is a hydrogen or an alkyl group having from about 1 to about 30 carbons; and Y² is selected from (CH₂)_{m″}, (CH₂CH₂O)_{m″}, or (CH₂CH₂CH₂O)_{m″} wherein m″ is an integer of from 1 to about 30. It is believed that, because of the alkyl group contained in the copolymer, the carboxylic acid/carboxylate copolymers do not make the composition undesirably sticky.

Suitable carboxylic acid/carboxylate copolymers herein are acrylic acid/alkyl acrylate copolymers having the following formula:

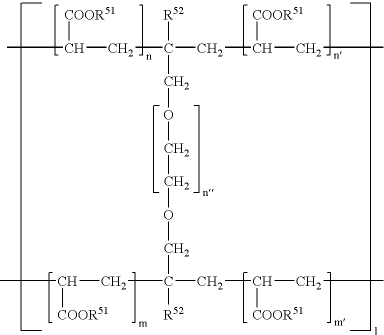

wherein R⁵¹, independently, is a hydrogen or an alkyl of 1 to 30 carbons wherein at least one; of R⁵¹ is a hydrogen, R⁵² is as defined above, n, n′, m and m′ are integers in which n+n′+m+m′ is from about 40 to about 100, n″ is an integer of from 1 to about 30, and l is defined so that the copolymer has a molecular weight of about 500,000 to about 3,000,000.

Commercially available carboxylic acid/carboxylate copolymers useful herein include: CTFA name Acrylates/C10-30 Alkyl Acrylate Crosspolymer having tradenames Pemulene TR-1, Pemulene TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

Surfactant System

The compositions of the present invention comprise from about 0.1% to about 2%, preferably from about 0.05% to about 1% of a surfactant system containing two or more nonionic surfactants selected from the group consisting of polyoxyalkylene alkyl ether having the C₁₂₋₁₈ of alkyl substitute, polyoxyalkylene hydrogenerated castor oil, and a liner or branched, mono- or tri-alkyl glyceride. The weight ratio of the surfactant system to the emollient oil is from about 2:1 to about 1:1.

The hydrophilic-lipophilic balance (HLB) of the surfactant system is selected by the artisan in view of the amount and species of the silicone component and the emollient oil. Preferably, the HLB of the surfactant system as a whole is higher than about 10. Preferably, among the surfactants used for the surfactant system, at least one surfactant has an HLB of more than 10, and at least one surfactant has an HLB of less than 10. More preferably, the amount of higher HLB surfactants is greater than the lower HLB surfactants. Without being bound by theory, by using both a higher HLB surfactant and a lower HLB surfactant, a stable emulsion can be made with as low level of surfactant possible. In a particularly preferred embodiment, at least 3 surfactants are used, such surfactants each having different HLBs.

In a preferred method of preparing the composition of the present invention, the surfactant system is first mixed with the emollient oil, and such mixture is added to water. The amount of water used in this step is preferably from about 0.5 to about 5 times, more preferably from about 1 to about 2.5 times the total weight of the emollient oil and surfactant system. Without being bound by theory, by incorporating the surfactant system in such manner, a stable emulsion can be made with as low level of surfactant possible.

Polyoxyalkylene alkyl ethers useful herein are the condensation products of alkylene oxides with both fatty acids and fatty alcohols (e.g., wherein the polyalkylene oxide portion is esterified on one end with a fatty acid and etherified (e.g., connected via an ether linkage) on the other end with a fatty alcohol). These materials have the general formula R¹CO(X₁)zOR² wherein R¹ and R² are independently alkyl of from about 12 to about 18 carbons; X₁ is —OCH₂CH₂ derived from, for example ethylene glycol or —OCH₂CHCH₃— derived from propylene glycol or oxide; and z is an integer from about 6 to about 50.

Nonlimiting examples of such alkylene oxide derived nonionic surfactants include ceteth-6, ceteth-10, ceteth-12, ceteareth-6, ceteareth-10, ceteareth-12, ceteareth-20, ceteareth-30, steareth-6, steareth-10, steareth-12, steareth-20, PEG-100 steareth, PEG-6 stearate, PEG-10 stearate, PEG-12 stearate, PEG-100 stearate, PEG-10 glyceryl stearate, PEG-20 glyceryl stearate, PEG-30 glyceryl cocoate, PEG-80 glyceryl cocoate, PEG-80 glyceryl tallowate, PEG-200 glyceryl tallowate, PEG-8 dilaurate, PEG-10 distearate, and mixtures thereof; preferably, ceteareth-12, ceteareth-20, and ceteareth-30. Commercially available surfactants include ceteareth-12 with tradename Eumulgin B1, ceteareth-20 with tradename Eumulgin B2, and ceteareth-30 with tradename Eumulgin B3, all available from Henkel.

Examples of polyoxyalkylene hydrogenerated castor oil useful herein include polyethylene hydrogenerated caster oil; preferably polyethylene (20) hydrogenerated caster oil.

Examples of mono- or tri-alkyl glyceride useful herein include glyceryl monostearate, glyceryl oleate, and triglyceryl diisostearate; preferably triglyceryl diisostearate.

Silicone Component

The compositions of the present invention comprise from about 0.05% to about 5%, preferably from about 0.5% to about 3% of a silicone component. The silicone components useful herein include volatile or nonvolatile insoluble silicones suitable for use on the skin. By insoluble what is meant is that the silicone forms a separate, discontinuous phase from the carrier, such as in the form of an emulsion or a suspension of droplets of the silicone. The silicone components herein may be made by any suitable method known in the art, including emulsion polymerization. The silicone components may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof. Silicone components of high molecular weight may be made by emulsion polymerization.

Silicone components useful herein include polyalkyl polyaryl siloxanes, silicone resins, amino-substituted siloxanes, and mixtures thereof. The silicone component is preferably selected from the group consisting of polyalkyl polyaryl siloxanes, silicone resins, and mixtures thereof, and more preferably from one or more polyalkyl polyaryl siloxanes.

Polyalkyl polyaryl siloxanes useful here in include those with the following structure (I)

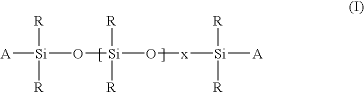

wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the skin, is compatible with the other components of the composition, is chemically stable under normal use and storage conditions, and is capable of being deposited on and conditions the skin. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described:by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Also useful are silicone resins, which are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone components on the skin.

The method of manufacturing these silicone components, can be found in Encyclopedia of Polymer Science and Engineering, Volume 15, Second Edition, pp. 204-308, John Wiley & Sons, Inc., 1989.

In one preferred embodiment, the silicone component is a mixture of high viscosity silicone compounds and silicone based carriers.

High viscosity silicone compounds herein include those having a molecular weight of from about 200,000 to about 540,000 selected from those mentioned above, preferably selected from the group consisting of dimethiconol, fluorosilicone dimethicone, and mixtures thereof, more preferably essentially dimethiconol. Particularly preferred dimethiconols are those having dimethylpolysiloxane repeating units, and terminated with hydroxy groups, wherein the dimethylsiloxane portion is made of from about 2700 to about 4500 repeating units.

Silicone based carriers include those having a viscosity of from about 0.65 mPa∘s to about 100 mPa∘s selected from cyclomethicones and dimethicones having lower repeating units.

Commercially available silicone components which are useful herein include Dimethicone with tradename DC345 available from Dow Corning Corporation, Dimethicone gum solutions with tradenames SE 30, SE 33, SE 54 and SE 76 available from General Electric, Dimethiconol with tradenames DCQ2-1403 and DCQ2-1401 available from Dow Corning Corporation, and emulsion polymerized Dimethiconol available from Toshiba Silicone as described in GB application 2,303,857.

Emollient Oil

The composition of the present invention comprises from about 0.5% to about 5%, preferably from about 1% to about 3% of an emollient oil. The emollient oil useful herein are those having a melting point of not more than about 25° C., and provide emollient benefit to the skin. The emollient oils useful herein may be volatile or nonvolatile, and include esters and hydrocarbons. Emollient oils of lower viscosity, low molecular weight, or branched structure, are highly preferable. It has been surprisingly found that, by the use of such oils, the tacky and greasy feel to the skin can be alleviated.

Emollient oils useful herein are esters, particularly esters having branched alkyl and alkenyl groups, for example, cetyl 2-ethyl hexyl, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, myristyl myristate, isostearyl palmitate, isocetyl palmitate, isodecyl palmitate, isopropyl palmitate, isostearyl myristate, octyl palmitate, caprylic/capric acid triglyceride, glyceryl tri-2-ethylhexanoate, neopentyl glycol di(2-ethyl hexanoate), neopentyl glycol dicaprate, diisopropyl dimerate, glycerol trioctanate, glycerol triisopalmitate, isopropyl myristate, octyldodecyl lactate, and mixtures thereof. Triglycerides such as caprylic/capric triglyceride, PEG-6 caprylic/capric triglyceride, and PEG-8 caprylic/capric triglyceride may also be useful. Crude mixtures of such triglycerides by the CTFA name Meadowfoam seed oil is also useful. Commercially available oils include, for example, isononyl isononanoate with tradenames Salacos 99 available from Nisshin Oil Mills, or Lanol 99 available from Seppic; tridecyl isononanoate with tradename Crodamol TN available from Croda, and Hexalan available from Nisshin Seiyu, and Meadowfoam Seed Oil with tradename Cropure MDF available from Croda.

Emollient oils also useful herein are the various grades and types of hydrocarbons. Mineral oils are liquid mixtures of hydrocarbons that are obtained from petroleum. Specific examples of suitable hydrocarbons include paraffin oil including those called light paraffin or isoparaffin, mineral oil, squalane, dodecane, isododecane, hexadecane, isohexadecane, eicosene, isoeicosene, tridecane, tetradecane, hydrogenated polyisobutylene, docosane, and mixtures thereof. Commercially available hydrocarbons useful herein include isododecane, isohexadeance, and isoeicosene with tradenames PERMETHYL 99A, PERMETHYL 101A, and PERMETHYL 1082, available from Presperse (South Plainfield N.J., USA), mineral oil with tradename BENOL available from Witco, isoparaffin with tradename ISOPAR available from Exxon Chemical Co. (Houston Tex., USA.), tradename Isoparaffin 2028 available from Idemitsu, and tradename Amsco Mineral Spirits available from Ashland.

Sebum Absorbing Agent

The composition of the present invention comprises from about 0.1% to about 10%, preferably from about 1% to about 5% of a sebum absorbing agent. Sebum absorbing agents useful herein include those which actually absorb the sebum discreted from the pores, and are compatible with the aqueous composition of the present invention. Components which are water soluble, water swellable, or have high emulsifying ability are not suitable herein, as they would no longer have sebum absorbing ability when formulated in the composition.

Preferable sebum absorbing agents herein include porous spherical cellulose powder, solid silicone elastomer powder, surface modified porous silica powder, porous nylon powder, porous acrylate copolymer, and mixtures thereof. The type and amount of sebum absorbing agents are selected according to the desired character of the product.

Preferred are porous spherical cellulose powders as disclosed in JP 61-100514 A and JP 61-189210 A publications. These materials are preferable in that they have good dispersibility in the present composition, and they provide a desirable skin feel upon use. The porous spherical cellulose powders preferred herein have an average particle size of from about 3 μm to about 50 μm, and do not include particles having a size of more than about. 100 μm. Any cellulose powder having the required, sebum absorbing ability and particle size can be used. Particularly useful porous spherical cellulose powders are those which are made by the following methods:

(1) Dispersing a copper (II) hydroxide ammonium solution of cellulose in organic solvents such as benzene to form spheres, treating such with acid, and obtaining the cellulose.

(2) Providing an organic solvent solution of cellulose ester, obtaining filaments of cellulose esters from such solution, cutting such filaments to provide chips, heat melting such chips to provide spherical particles, and saponifying such particles.

(3) Dispersing the solution as mentioned in (2) in a hardly soluble or sparingly soluble organic solvent, heating such dispersion to remove the organic solvent to obtain spherical particles, and saponifying such particles.

The cellulose esters that can be used in methods (2) and (3) above include: partial, complete, and mixed esters of cellulose with acetic acid, proprionic acid, butyric acid, nitric acid, and others. Among these esters, cellulose triacetate is preferred.

Commercially available porous spherical cellulose powders highly useful herein include the materials with tradename Celluflow series, such as Celluflow C025 available from Chisso Corp.

Preferred are solid silicone elastomer powders which are fine particles of a silicone rubber of which the particles have a composite structure as disclosed in EP 661,334 A publication. These materials are preferable in that they have provide a smooth feel upon use. These fine particles of a silicone rubber of which the particles have a composite structure consist of a spherical or globular particle of a cured silicone rubber having an average particle diameter in the range from 0.1 to 50 μm and a coating layer of a polyorganosilsesquioxane resin, the coating amount of the polyorganosilsesquioxane resin being in the range from 1 to 500 parts by weight per 100 parts by weight of the silicone rubber particles before coating.

The above defined silicone resin-coated silicone rubber particles can be prepared by a method which comprises the step of: admixing an aqueous dispersion of particles of a cured silicone rubber having an average particle diameter in the range from 0.1 to 50 μm with an alkaline compound and a trialkoxy silane compound represented by the general formula R²Si(OR¹)₃, in which R2 is an unsubstituted or substituted monovalent hydrocarbon group having 1-20 carbon atoms and R1 is an alkyl group having 1 to 6 carbon atoms, in combination at a temperature not exceeding 60° C. under agitation.

These fine particles are characterized by the composite structure of the particles consisting of a cured silicone rubber particle having a specified average particle diameter and a coating layer thereon formed from a polyorganosilsesquioxane resin in a specified coating amount and the coated silicone rubber particles can be prepared by the in situ hydrolysis and condensation reaction of a trialkoxy silane compound in the presence of cured silicone rubber particles in an aqueous dispersion so as to form the coating layer of a polyorganosilsesquioxane resin on the surface of the silicone rubber particles.

The silicone rubber forming the fine core particles, on which the coating layer of the polyorganosilsesquioxane resin is formed, is a cured diorganopplysiloxane having linear diorganopolysiloxane segments represented by the general formula in which each R is, independently from the others, an unsubstituted or substituted monovalent hydrocarbon group having 1 to 20 carbon atoms exemplified by alkyl groups such as methyl, ethyl, propyl and butyl groups, aryl groups such as phenyl and tolyl groups, alkenyl groups such as vinyl and allyl groups and aralkyl groups such as 2-phenylethyl and 2-phenylpropyl groups as well as those substituted hydrocarbon groups obtained by replacing a part or all of the hydrogen atoms in the above named hydrocarbon groups with substituents including halogen atoms, epoxy group, amino group, mercapto group, (meth)acryloxy group and the like such as chloromethyl and 3,3,3-trifluoropropyl groups, at least 90% by moles of the groups R being preferably methyl groups, and the subscript a is a positive integer in the range, though not particularly limitative, from 5 to 5000 or, preferably, from 10 to 1000. Such value of the subscript is selected to that the silicone rubber particles after coating with the silicone resin can impart the matrix material compounded therewith with an adequate internal stress and improved surface lubricity while, difficulties are not encountered in the preparation of silicon rubber particles. It is optional that the silicone rubber forming the fine particles is compounded with a silicone oil, organosilane compound, inorganic and organic powders and the like.

It is particularly useful to have the silicone rubber particles coated with a coating layer of the silicone resin have an average particle diameter in the range from 0.1 to 50 μm or, preferably, from 1 to 20 μm. Such average particle diameter of the silicone rubber particles is advantageous for providing suitable flowability of the particles without increased agglomeration while, not affecting surface lubricity of the shaped articles of a composition compounded with the silicone resin-coated silicone rubber particles, or physical properties of the shaped articles.

Commercially available solid silicone elastomer powders highly useful herein include vinyl dimethicone/methicone silsesquioxane crosspolymer with tradenames KSP series available from ShinEtsu Chemical Co., Ltd., Tokyo Japan.

Other commercially available sebum absorbing agents, include porous acrylate copolymers with tradename Polytrap available from Dow Corning.

Water Soluble Humectant

The composition of the present invention comprises from about 1% to about 20%, preferably from about 5% to about 15% of a water soluble humectant. Water soluble humectants useful herein include polyhydric alcohols such as glycerin, sorbitol, propylene glycol, butylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants herein include: glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; polyethylene glycols with the tradename CARBOWAX series available from Union Carbide, and a mixture of glyceryl polymethacrylate, propylene glycol and PVM/MA copolymer with tradename Lubrajel Oil available from Guardian Lab.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product.

The pH of the present composition is preferably from about 4 to about 8, more preferably from about 5 to about 7. The suitable tacky skin treatment agents are particularly efficient in such pH range. Buffers and other pH adjusting agents can be included to achieve the desirable pH.

Additional Water Soluble Polymer

The compositions of the present invention may further comprise an additional water soluble polymer, preferably at a level of from about 0.01% to about 5%, more preferably from about 0.04% to about 1%. The additional water soluble polymers herein are water soluble or water miscible polymers, and are compatible with the carboxylic acid/carboxylate copolymers. Without being bound by theory, it is believed the controlled amount of additional water soluble polymers in the composition provides improved moisturization and smoothness to the skin without giving an undesirable tacky or sticky feeling.

In one preferred embodiment, the additional water soluble polymer is selected so that the composition of the present composition has a suitable viscosity of preferably from about 100 mPa·s to about 6000 mPa·s, as described above.

Additional water soluble polymers useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxyethyl ethylcellulose, hydroxypropyl methyl cellulose, nitro cellulose, sodium cellulose sulfate, sodium carboxymethyl cellulose, crystalline cellulose, cellulose powder, pullulan, mannan, scleroglucans, polyvinylpyrrolidone, polyvinyl alcohol, guar gum, hydroxypropyl guar gum, xanthan gum, acacia gum, arabia, gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Polyalkylene glycols having a molecular weight of more than about 1000 are useful herein. Useful are those having the following general formula:

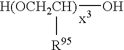

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers,. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as Polyox WSR® N-3000 available from Union Carbide).

Commercially available additional water soluble polymers highly useful herein include xanthan gum with tradename Keltrol series available from Kelco, Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Herculus, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Additional water soluble polymers useful herein include amphoteric polymers. The amphoteric polymers useful herein are those including at least one cationic monomer and at least one anionic monomer; the cationic monomer being quaternary ammonium, preferably dialkyl diallyl ammonium chloride or carboxylamidoalkyl trialkyl ammonium chloride; and the anionic monomer being carboxylic acid. The amphoteric conditioning polymers herein may include nonionic monomers such as acrylamine, methacrylate, or ethacrylate.

Useful herein are polymers with the CTFA name Polyquaternium 22, Polyquaternium 39, and Polyquaternium 47. Such polymers are, for example, copolymers consisting of dimethyldiallyl ammonium chloride and acrylic acid, terpolymers consisting of dimethyldiallyl ammonium chloride and acrylamide, and terpolymers consisting of acrylic acid methacrylamidopropyl trimethylammonium chloride and methyl acrylate such as those of the following formula wherein the ratio of n⁶:n⁷:n⁸ is 45:45:10:

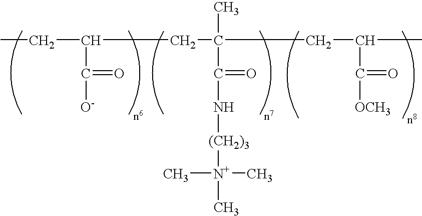

Highly preferred commercially available amphoteric polymers herein include Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation.

Also useful herein are polymers resulting from the copolymerisation of a vinyl monomer carrying at least one carboxyl group, such as acrylic acid, methacrylic acid, maleic acid, itaconic acid, fumaric acid, crotonic acid, or alphachloroacrylic acid, and a basic monomer which is a substituted vinyl compound containing at least one basic nitrogen atom, such as dialkylaminoalkyl methacrylates and acrylates and dialkylaminoalkylmethacrylamides and acrylamides.

Also useful herein are polymers containing units derived from:

i) at least one monomer chosen from amongst acrylamides or methacrylamides substituted on the nitrogen by an alkyl radical,

ii) at least one acid comonomer containing one or more reactive carboxyl groups, and

iii) at least one basic comonomer, such as esters, with primary, secondary and tertiary amine substituents and quaternary ammonium substituents, of acrylic and methacrylic acids, and the product resulting from the quaternisation of dimethylaminoethyl methacrylate with dimethyl or diethyl sulfate.

The N-substituted acrylamides or methacrylamides which are most particularly preferred are the groups in which the alkyl radicals contain from 2 to 12 carbon atoms, especially N-ethylacrylamide, N-tert.-butylacrylamide, N-tert.-octylacrylamide, N-octylacrylamide, N-decylacrylamide and N-dodecylacrylamide and also the corresponding methacrylamides. The acid comonomers are chosen more particularly from amongst acrylic, methacrylic, crotonic, itaconic, maleic and fumaric acids and also the alkyl monoesters of maleic acid or fumaric acid in which alkyl has 1 to 4 carbon atoms.

The preferred basic comonomers are aminoethyl, butylaminoethyl, N,N′-dimethylaminoethyl and N-tert.-butylaminoethyl methacrylates.

Commercially available amphoteric conditioning polymers herein include octylacrylamine/acrylates/butylaminoethyl methacrylate copolymers with the tradenames AMPHOMER, AMPHOMER SH701, AMPHOMER 28-4910, AMPHOMER LV71, and. AMPHOMER LV47 supplied by National Starch & Chemical.

Tacky Skin Treatment Agent

The composition of the present invention may further comprise from about 0.5% to about 10%, preferably from about 1% to about 5% of a tacky skin treatment agent. Skin treatment agents useful herein are those which help repair and replenish the natural moisture barrier function of the epidermis, thereby providing skin benefits such as texture improvement. It is generally known that, while such agents provide useful benefits to the skin when used chronically, they also tend to provide negative skin feel upon use when applied by itself.

Tacky skin treatment agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, urea, gelatin, soluble collagen, royal jelly, tocopheryl nicotinate, and vitamin D3 and analogues or derivatives, and mixtures thereof. Niacinamide is particularly preferred in that, when used in a pharmaceutically effective amount, is capable of reducing or alleviating the intensity of chronical spots. Niacinamide is suitably incorporated in the composition by first dissolving in water. Panthenol is also particularly preferred in that, when used in an amount of at least about 1%, it provides texture improvement benefits. Niacinamide and panthenol are commercially available, for example, by Roche.

Sebum Supressing Plant Extract

The composition of the present invention may further comprise from about 0.001% to about 5%, more preferably from about 0.05% to about 1% of a sebum suppressing plant extract. The plant extracts useful herein are those which have an astringent type of effect for reducing the size of pores, or inhibition effect of 5-α-reductase, and are compatible with the aqueous form of the present composition, and preferably do not alter the transparent or translucent appearance of the present composition. Water soluble plant extracts are preferred. Useful plant extracts herein include clove (choji) extract, coix (yokuinin) extract, witch hazel (hamamerisu) extract, and mixtures thereof. Such plant extracts are available from Iwase.

UV Protecting Agent

The composition of the present invention may further comprise from about 1% to about 10%, more preferably from about 3% to about 9.5% of a UV protecting agent. UV protecting agents generally prevent excessive scaling and texture changes of the stratum corneum by exposure of ultraviolet light and may be added to the emulsion of the present invention. Suitable UV protecting agents may be organic or inorganic. Hydrophobic and water insoluble UV protecting agents may be used at controlled levels.

A wide variety of conventional UV protecting agent are suitable for use herein. See, U.S. Pat. No. 5,087,445, Haffey et al., issued Feb. 11, 1992; U.S. Pat. No. 5,073,372, Turner et al., issued Dec. 17, 1991; U.S. Pat. No. 5,073,371, Turner et al., issued Dec. 17, 1991; and Segarin, et al., at Chapter VIII, pages 189 et seq., of Cosmetics Science and Technology (1972), which discloses numerous suitable UV protecting agent. Preferred among those UV protecting agent which are useful in the emulsions are those selected from 2-ethylhexyl-p-methoxycinnamate (commercially available as PARSOL MCX), butylmethoxydibenzoylmethane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide such as MT-100 available from Tayca, zinc oxide, silica, iron oxide, Eusolex™ 6300, Octocrylene, Parsol 1789, and mixtures thereof.

Particularly useful herein are UV protecting agents such as those disclosed in U.S. Pat. No. 4,937,370, Sabatelli, issued Jun. 26, 1990, and U.S. Pat. No. 4,999,186, Sabatelli, issued Mar. 12, 1991. The UV protecting agent disclosed therein have, in a single molecular, two distinct chromophore moieties which exhibit different ultraviolet radiation absorption spectra. One of the chromophore moieties absorbs predominantly in the UVB radiation range and the other absorbs strongly in the UVA radiation range. These UV protecting agent provide higher efficacy, broader UV absorption, lower skin penetration and longer lasting efficacy relative to conventional UV protecting agent.

Exact amounts will vary depending upon the sunscreen chosen and the desired Sun Protection Factor (SPF). SPF is a commonly used measure of photoprotection of a sunscreen. against erythema. See Federal Register, Vol. 43, No. 166, pp. 38206-38269, Aug. 25, 1978.

Whitening Agent

The composition of the present invention may further comprise from about 0.001% to about 10%, more preferably from about 0.1% to about 5% of a whitening agent. Whitening agents useful herein are those which are compatible with the aqueous form of the present composition. Water soluble whitening agents are preferred. The whitening agent useful herein refers to active ingredients that not only alter the appearance of the skin, but further improve hyperpigmentation as compared to pre-treatment.

Useful whitening agents useful herein include ascorbic acid compounds, azelaic acid, butyl hydroxy anisole, gallic acid and its derivatives, glycyrrhizinic acid, hydroquinoine, kojic acid, arbutin, mulberry,extract, and mixtures thereof. Use of combinations of whitening agents are believed to be advantageous in that they may provide whitening benefit through different. mechanisms.

Ascorbic acid compounds are useful whitening agents, and have the formula (I):

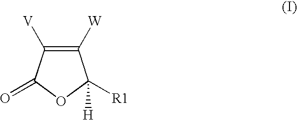

wherein V and W are independently —OH; R¹ is —CH(OH)—CH₂OH; and salts thereof.

Preferably, the ascorbic acid compound useful herein is an ascorbic acid salt or derivative thereof, such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art.

More preferably, the ascorbic acid salt useful herein is a metal ascorbate having the following formula (II):

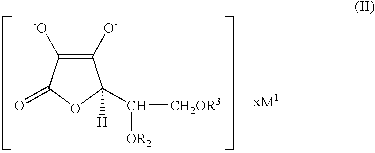

wherein R² and R³ are independently selected from hydrogen and linear or branched alkyl of 1 to about 8 carbons; M¹ is a metal; and x is an integer of from 1 to about 3. More preferably, R² and R³ are independently selected from hydrogen and linear or branched alkyl of 1 to about 3 carbons; M¹ is sodium, potassium, magnesium, or calcium.

Examples of other preferred ascorbic acid salts having formula (II) include monovalent metal salts (e.g., sodium ascorbate, potassium ascorbate), divalent metal salts (e.g., magnesium ascorbate, calcium ascorbate) and trivalent metal salts (e.g., aluminum ascorbate) of ascorbic acid.

Preferably, the ascorbic acid salt useful herein is a water soluble ascorbyl ester having; the following formula (III):

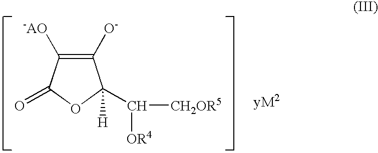

wherein A is sulfate or phosphate; R⁴ and R⁵ are independently selected from hydrogen and linear or branched alkyl of 1 to about 8 carbons; M² is a metal; and y is an integer of 1 to about 3. More preferably, R⁴ and R⁵ are independently selected from hydrogen and linear or branched alkyl of 1 to about 3 carbons; M² is sodium, potassium, magnesium, or calcium.

Another particularly preferred ascorbic acid compound is 2-o-α-D-glucopyranosyl-L-ascorbic acid, usually referred to as L-ascorbic acid 2-glucoside or ascorbyl glucoside, and its metal salts. Such compounds are available from Hayashibara.

Exemplary water soluble salt derivatives include, but are not limited to, L-ascorbic acid 2-glucoside, L-ascorbyl phosphate ester salts such as sodium L-ascorbyl phosphate, potassium L-ascorbyl phosphate, magnesium L-ascorbyl phosphate, calcium L-ascorbyl phosphate, aluminum L-ascorbyl phosphate. L-ascorbyl sulfate ester salts can also be used. Examples are sodium L-ascorbyl sulfate, potassium L-ascorbyl sulfate, magnesium L-ascorbyl sulfate, calcium L-ascorbyl sulfate and aluminum L-ascorbyl sulfate.

Additional Components

The compositions herein may further contain other additional components, which may be selected by the artisan according to the desired characteristics of the final product and which are suitable for rendering the compositions more cosmetically or aesthetically acceptable or to provide them with additional usage benefits. The components useful herein are conveniently categorized by a certain benefit or their postulated mode of action, however, a given category is not limiting of its use. Further, it is understood the one component may provide multiple benefits.

(i) Anti-Oxidants and Radical Scavengers

Anti-oxidants and radical scavengers are especially useful for providing protection against UV radiation which can cause increased scaling or texture changes in the stratum corneum and against other environmental agents which can cause skin damage.

Anti-oxidants and radical scavengers such as tocopherol (vitamin E), tocopherol sorbate, tocopherol acetate, other esters of tocopherol, propyl gallate, alkyl esters of uric acid, amines (i.e., N,N-diethylhydroxylamine, amino-guanidine), sulfhydryl compounds (i.e., glutathione), lycine pidolate, arginine pilolate, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol; sorbate. For example, the use of tocopherol sorbate in topical emulsions and applicable to the present invention is described in U.S. Pat. No. 4,847,071, Bissett et al., issued Jul. 11, 1989.

(ii) Anti-Inflammatory Agents

Anti-inflammatory agents enhance the skin appearance benefits, by for example, contribution of uniformity and acceptable skin tone and/or color.

Preferably, the anti-inflammatory agent includes a steroidal anti-inflammatory agent and an non-steroidal anti-inflammatory agent. Preferred steroidal anti-inflammatory for use is hydrocortisone.

The variety of compounds encompassed by this group are well-known to those skilled in the art. For detailed disclosure of the chemical structure, synthesis, side effects, etc. of non-steroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974), each incorporated herein by reference.

So-called “natural” anti-inflammatory agents are also useful. Such agents may suitably be obtained as an extract by suitable physical and/or chemical isolation from natural sources (i.e., plants, fungi, by-products of microorganisms). For example, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

(iii) Antimicrobial Agent

As used, “antimicrobial agents” means a compound capable of destroying microbes, preventing the development of microbes or preventing the pathogenic action of microbes. Antimicrobal agents are useful, for example, in controlling acne. Preferred antimicrobial agents useful in the present invention are benzoyl peroxide, erythromycin, tetracycline, clindamycin, azelaic acid, sulfur resorcinol, phenoxyethanol, and Irgasan™ DP 300 (Ciba Geigy Corp., U.S.A.). A safe and effective amount of an antimicrobial agent may be added to emulsions of the present invention, preferably from about 0.001% to about 10%, more preferably from about 0.01% to about 5%, still more preferably from about 0.05% to about 2%.

(iv) Chelators

As used herein, “chelator” refers to a compound that reacts for removing a metal ion from a system by forming a complex so that the metal ion cannot readily participate in or catalyze chemical reactions. The inclusion of a chelator is especially useful for providing protection against UV radiation which can contribute to excessive scaling or skin texture changes and against other environmental agents which can cause skin damage.

Exemplary chelators that are useful herein are disclosed in U.S. Pat. No. 5,487,884, Bissett et al., issued Jan. 30, 1996; PCT application 91/16035 and 91/16034, Bush et al., published Oct. 31, 1995. Preferred chelators are furildioxime and derivatives thereof.

(v) Other Components

In addition to the above described components, the composition of the present invention may further include preservatives and preservative enhancers such as water-soluble or solubilizable preservatives including Germall 115, methyl, ethyl, propyl and butyl esters of hydroxybenzoic acid, benzyl alcohol, imidazolidinyl urea, EDTA and its salts, Bronopol (2-bromo-2-nitropropane-1,3-diol) and phenoxypropanol; antifoaming agents; binders; biological additives; bulking agents; coloring agents; perfumes, essential oils, and solubilizers thereof; other natural extracts; compounds which stimulate collagen production; yeast fermented filtrates, and others.

Method of Preparation

The present invention also relates to a method of preparing a stable transparent composition of aqueous base containing silicone components and emollient oils. The present method is useful for making what is generally known as a microemulsion, as explained above under section “Composition”. The base composition made by the present method has such transparency that the composition has an absorbance of less than about 2 at a wave length of 340 nm, preferably from about 1 to about 1.5 at the same wave length. When sebum absorbing agents are included in the present composition, the composition may become less transparent.

The present method comprises the steps of:

(a) preparing a first mixture comprising the steps of:

(a1) mixing an emollient oil with a surfactant system and water at a temperature of about 70-80° C.; the amount of such water being from about 0.5 to about 5 times the total weight of the emollient oil and surfactant system;

(a2) cooling the product of (a1) to about 50° C.;

(a3) adding the product of (a2) to water, the temperature of such water being cold enough to immediately cool the obtained mixture to a temperature; of no higher. than 40° C.;

(b) preparing a second mixture comprising the steps of:

(b1) dispersing a carboxylic acid/carboxylate copolymer into water at a temperature of about 70-80° C. and mixing until homogeneous;

(b2) adding a silicone component to the product of step (b1); and

(c) mixing the first mixture and the second mixture;

wherein the surfactant system comprises two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides.

The composition to be made by the present method comprises at least: a carboxylic acid/carboxylate copolymer, a surfactant system, a silicone component, an emollient oil, and an aqueous carrier. While not essential to the present method, a water soluble humectant is typically added. The components used in the present method are the same as those described above.

A first mixture is prepared with the specific surfactant system, emollient oil, and water. In step (a1), the emollient oil, the surfactant system, and water are mixed together in an anchor mixer at a temperature of about 70-80° C. to form an emulsion. Preferably, the surfactant system is first mixed with the emollient oil, and such mixture is added to water. The amount of water used in step (a1) is from about 0.5 to about 5 times, preferably from about 1 to about 2.5 times the total weight of the emollient oil and surfactant system. In step (a2), the product of step (a1) is cooled to 50° C., preferably within a period of about 20 minutes., It has been discovered that such cooling allows the mixture to become transparent. In step (a3), the product of step (a2) is further cooled by mixing with cold water. The cold water to be mixed with the product of step (a2) has a temperature cold enough that the obtained mixture is immediately cooled to no higher than 40° C., preferably about 30° C. Without being bound by theory, it is believed this second cooling process provides a stable transparent mixture with as small amount of surfactant as possible. Water soluble humectant and preservatives may be added to the cold water prior to step (a3).

Separately, a second mixture is prepared with the carboxylic acid/carboxylate copolymer, the silicone component, and water. In step (b1), the carboxylic acid/carboxylate copolymer, and if present, additional water soluble polymers are dispersed in water at a temperature of about 70-80° C. until homogeneous. The amount of water to be used in step (b1) is selected by the artisan. A high speed agitator may be used. The rotation speed is controlled to no more than about 5000 rpm to avoid destruction of the polymer structures. When sebum absorbing agents and/or neutralizing agents are present, they are added subsequent to this step. In step (b2), the product of step (b1) is further mixed with the silicone component. The remaining components, if present, such as water soluble humectants, tacky skin treatment agents, sebum suppressing plant extracts, UV absorbing agents, and whitening agents, may be added to the product of step (b2).

Finally, the first mixture and the second mixture is mixed until homogeneous (Step c). Mixing at this step is preferably conducted by an anchor mixer at a relatively low rotation speed, preferably from about 50 rpm to about 100 rpm. This step can be carried out at room temperature.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Compositions

| Phase | | Ex. 1 | Ex. 2 | Ex. 3 | Ex. 4 | Ex. 5 | Ex. 6 |
|---|---|---|---|---|---|---|---|
| 01 | Ceteareth-12 *1 | 0.015 | 0.1 | 0.1 | 0.1 | 0.1 | 0.1 |
| 01 | Ceteareth-20 *2 | 0.05 | 0.1 | 0.1 | 0.2 | 0.2 | 0.2 |
| 01 | Ceteareth-30 *3 | 0.09 | 0.2 | 0.2 | 0.2 | 0.2 | 0.25 |
| 01 | Polyglyceryl-3 Diisostearate | 0.1 | 0.2 | 0.2 | 0.1 | 0.1 | |
| 01 | PEG-20 Hydrogenated Castor Oil | 0.04 | 0.05 | 0.05 | 0.1 | 0.1 | 0.2 |
| 01 | Cetyl Octanoate | 0.1 | | | | | 0.1 |
| 01 | Meadowfoam Seed Oil *4 | 0.1 | 0.05 | | 0.1 | | |
| 01 | Isohexadecane *5 | | | | 0.1 | 0.1 | 0.1 |
| 01 | Isododecane *6 | | 0.1 | | | | |
| 01 | Isononyl Isononanoate *7 | | | 0.1 | | | |
| 02 | De ionized Water | 2.0 | 5.0 | 4.0 | 4.0 | 4.0 | 4.0 |
| 03 | De ionized Water | 30.0 | 30.0 | 30.0 | 30.0 | 30.0 | 30.0 |
| 03 | 1,3-Butylene glycol | 2.0 | 2.0 | 2.0 | 2.0 | 2.0 | 2.0 |
| 03 | Methyl Paraben | 0.07 | 0.07 | 0.07 | 0.07 | 0.07 | 0.07 |
| 03 | Sodium Benzoate | 0.07 | 0.07 | 0.07 | 0.07 | 0.07 | 0.07 |
| 04 | Acrylic acid alkyl acrylate | | | | | 0.24 | 0.2 |
| | copolymer 1 *8 | | | | | | |
| 04 | Acrylic acid alkyl acrylate | 0.25 | 0.24 | 0.24 | 0.3 | | |
| | copolymer 2 *9 | | | | | | |
| 04 | Xanthan gum *10 | 0.04 | 0.04 | 0.04 | 0.04 | | |
| 04 | Carbomer *11 | | | | | 0.2 | |
| 04 | Sodium Hyaluronate *12 | 0.05 | | | | 0.02 | 0.02 |
| 04 | De ionized Water | balance to 100% | | | | | |
| 05 | Sodium Hydroxide | 0.07 | 0.1 | 0.1 | | 0.2 | |
| 05 | Aminomethyl propanol | | | | 0.12 | 0.15 | 0.1 |
| 06 | Panthenol *13 | 1.0 | 1.0 | 1.0 | 1.0 | 2.0 | 1.0 |
| 06 | Niacinamide *14 | 2.0 | 2.0 | 2.0 | 2.0 | 2.0 | 2.0 |
| 06 | Glycerin | 2.5 | 2.5 | 2.5 | 2.5 | 3.0 | 4.0 |
| 06 | 1,3-butylene glycol | 5.5 | 4.0 | 2.0 | 7.0 | 4.0 | 4.0 |
| 06 | Glyceryl Polymethacrylate | 1.5 | | 1.5 | 2.0 | | |
| | & Propylene Glycol & | | | | | | |
| | PVM/MA copolymer *15 | | | | | | |
| 06 | Witch Hazel Extract *16 | 0.1 | | | | | |
| 06 | Clove Extract *17 | | | 0.1 | | | |
| 07 | Magnesium Ascorbyl Phosphate | | | | | 3.0 | |
| 07 | Benzophenone-1 | | | | | | 2.0 |
| 07 | Titanium Dioxide *18 | | | | | | 1.0 |
| 08 | Dimethicone/Dimethiconol *19 | 1.5 | | 2.0 | | 1.0 | |
| 08 | Cyclomethicone/Dimethiconol *20 | | 1.5 | | 1.5 | | 1.0 |
| 09 | Cellulose Powder *21 | 1.0 | 3.0 | | | 3.0 | |
| 09 | Vinyl Dimethicone/methicone | | | 3.0 | 5.0 | | 5.0 |
| | Silsesquioxane Crosspolymer *22 | | | | | | |
| 10 | Methyl Paraben | 0.2 | 0.2 | 0.2 | 0.2 | 0.2 | 0.2 |
| 10 | Benzyl alcohol | 0.3 | 0.3 | 0.3 | 0.3 | 0.3 | 0.3 |
| 10 | Sodium benzoate | 0.05 | 0.05 | 0.05 | 0.05 | 0.05 | 0.05 |
| 10 | EDTA-2Na | 0.1 | 0.1 | 0.1 | 0.1 | 0.1 | 0.1 |

Definitions of Components
*1 Ceteareth-12 (POE(12) Cetostearyl Ether): Eumulgin B1 available from Henkel
*2 Ceteareth-20 (POE(20) Cetostearyl Ether): Eumulgin B2 available from Henkel
*3 Ceteareth-30 (POE(30) Cetostearyl Ether): Eumulgin B3 available from Henkel
*4 Meadowfoam Seed Oil: Cropure MDF available from Croda
*5 Isohexadecane: Permethyl 101A available from Presperse
*6 Isododecane: Permethyl 99A available from Presperse
*7 Isononyl isononanoate: Salacos 99 available from Nisshin Oil Mills
*8 Acrylic acid/alkyl acrylate copolymer 1: PEMULEN TR-1 available from B. F. Goodrich
*9 Acrylic acid/alkyl acrylate copolymer 2: PEMULEN TR-2 available from B. F. Goodrich
*10 Xanthan gum: Keltrol T available from Kelco
*11 Carbomer: Carbopol 981 available from B. F. Goodrich
*12 Sodium Hyaluronate: available from Chisso corp.
*13 Panthenol: available from Roche
*14 Niacinamide: available from Roche
*15 Glyceryl Polymethacrylate & Propylene Glycol & PVM/MA copolymer: Lubrajel Oil available from Guardian Lab
*16 Witch Hazel Extract: Hamamerisu Liquid available from Iwase
*17 Clove Extract: Choji Extract BG available from Iwase
*18 Titanium Dioxide: Titanium Dioxide MT-100 available from Tayca
*19 Dimethicone/Dimethiconol: DCQ2-1403 available from Dow Corning
*20 Cyclomethicone/Dimethiconol: DCQ2-1401 available from Dow Corning
*21 Cellulose Powder: Celluflow C-25 available from Chisso corp.
*22 Vinyl dimethycon/methicone Silsesqueioxane Crosspolymer: KSP-100 available from Shinetsu Chemical

Method of Preparation

a1) Heat Phase 01 to about 80. Heat Phase 02 to about 78. Emulsify by adding Phase 01 to Phase 02 and mixing.

a2) Cool the product of a2) to about 50 within a period of about 10 to about 20 minutes.

a3) Add the product of a2) to Phase 03, Phase 03 being at room temperature.

b1) Using the components of Phase 04, provide a 2% solution of Acrylic acid/alkyl acrylate copolymer, 2% solution of xanthan gum if present, and 0.4% solution of sodium hyaluronate in water of about 80 , and mix such solutions with high speed agitator at no more than 5000 rpm, until homogenous. Add Phase 09 under mixing. Add Phase 05 under mixing to neutralize.

b2) Add Phase 08, and Phases 05, 06 and 07 if present, to the product of b1) with a high speed agitator. Add Phase 10.

c) Mix the product of step a3) and the product of step b2), and cool to below 40° C.

The embodiments disclosed and represented by the previous examples have many advantages. For example, Examples 1 through 6 are particularly useful for providing clear lotions for use on the facial skin. When used on the facial skin, the compositions of Examples 1 through 6 provide moisturizing benefit to the skin without leaving a tacky and/or greasy feel to the skin. When used daily on the facial skin over a period of at least 4 weeks, Examples 1 through 6 provide significant improvement of controlling excess sebum. Example 5 further provides chronic whitening benefit. Example 6 further provides UV protection benefit to the skin.

Examples 7 through 9 are made by using the same compositions of Examples 1, 2, and 4, respectively, but lacking the cellulose powder. Examples 10 through 12 are made by using the same compositions of Examples 3, 5, and 6, respectively, but lacking the vinyl dimethicone/methicone silsesqueioxane. Examples 7 through 12 have a transparent appearance, and an absorbance of no more than about 2 at a wave length of 340 nm.

What is claimed is:

1. A skin care composition comprising by weight:
(1) from about 0.01% to about 5% of a carboxylic acid/carboxylate copolymer;
(2) from about 0.1% to about 2% of a surfactant system comprising two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides;
(3) from about 0.05% to about 5% of a silicone component;
(4) from about 0.01% to about 5% of an emollient oil;
(5) from about 0. 1% to about 10% of a sebum absorbing agent;
(6) from about 1% to about 20% of a water soluble humectant; and
(7) an aqueous carrier;
wherein the weight ratio of the surfactant system to the emollient oil is from about 2:1 to about 1:1; and wherein a base composition consisting essentially of components (1) through (4), (6) and (7) above has an absorbance of no more than about 2 at a wave length of 340 nm.

2. The skin care composition according to claim 1 wherein the sebum absorbing agent is selected from the group consisting of porous spherical cellulose powder, solid silicone elastomer powder, surface modified porous silica powder, porous nylon powder, porous acrylate copolymer, and mixtures thereof.

3. The skin care composition according to claim 2 wherein the sebum absorbing agent is porous spherical cellulose powder.

4. The skin care composition according to claim 1 further comprising an additional water soluble polymer.

5. The skin care composition according to claim 1 further comprising a tacky skin treatment agent.

6. The skin care composition according to claim 1 further comprising a sebum suppressing plant extract.

7. The skin care composition according to claim 1 further comprising a UV protecting agent.

8. The skin care composition according to claim 1 further comprising a whitening agent.

9. A method of preparation of a composition comprising the steps of:
(a) preparing a first mixture comprising the steps of:
(a1) mixing an emollient oil with a surfactant system and water at a temperature of about 70-80° C.; the amount of such water being from about 0.5 to about 5 times the total weight of the emollient oil and surfactant system;
(a2) cooling the product of (a1) to about 50° C.;
(a3) adding the product of (a2) to water, the temperature of such water being cold enough to immediately cool the obtained mixture to a temperature of no higher than 40° C.;
(b) preparing a second mixture comprising the steps of:
(b1) dispersing a carboxylic acid/carboxylate copolymer into water at a temperature of about 70-80° C. and mixing until homogeneous;
(b2) adding a silicone component to the product of step (b1); and
(c) mixing the first mixture and the second mixture;
wherein the surfactant system comprises two or more nonionic surfactants selected from the group consisting of polyoxyalkylene alkyl ethers having a C12-18 alkyl substitute, polyoxyalkylene hydrogenated castor oils, and linear or branched, mono- or tri-alkyl glycerides.

10. The method of preparation according to claim 9 wherein the composition further comprises a sebum absorbing powder, the sebum absorbing powder added to the product of (b1) prior to step (b2).

11. A method of controlling excess sebum comprising the step of applying the composition of claim 1 to the skin.
